(19) United States
(12) Patent Application Publication
(10) Pub. No.: US 2009/0239957 A1
(43) Pub. Date: Sep. 24, 2009

(54) STABILIZED GLYCEROPHOSPHATE-CONTAINING SURGICAL IRRIGATING SOLUTION

(75) Inventors: Mandar V. SHAH, Rockaway, NJ (US); Joseph Paul BULLOCK, Fort Worth, TX (US); Alan L. WEINER, Arlington, TX (US); Harli DOLLINGER, Fort Worth, TX (US); Simaben B. PATEL, Fort Worth, TX (US)

Correspondence Address: ALCON, IP LEGAL, TB4-8, 6201 SOUTH FREEWAY, FORT WORTH, TX, 76134, US

(73) Assignee: ALCON RESEARCH, LTD., Fort Worth, TX (US)

(21) Appl. No.: 12/339,536

(22) Filed: Dec. 19, 2008

Related U.S. Application Data
(60) Provisional application No. 61/016,119, filed on Dec. 21, 2007.

(51) Int. Cl. A61K 47/02; A61K 47/04

(52) U.S. Cl. 514/770; 514/769

(57) ABSTRACT

One aspect of the present invention relates to stabilized irrigating compositions comprising sodium glycerophosphate and/or calcium glycerophosphate, and a bicarbonate salt. Another aspect of the present invention relates to methods of irrigating ocular tissues during a surgical procedure comprising bathing the intraocular tissues with an irrigating composition comprising sodium glycerophosphate and/or calcium glycerophosphate, and a bicarbonate salt.

### CROSS-REFERENCE TO RELATED APPLICATION

This application claims priority under 35 U.S.C. §119 to U.S. Provisional Patent Application No. 61/016,119, filed Dec. 21, 2007, the entire contents of which are incorporated herein by reference.

### TECHNICAL FIELD OF THE INVENTION

The present invention relates generally to surgical irrigating solutions and more specifically to improved ophthalmic surgical irrigating solutions.

### BACKGROUND OF THE INVENTION

A wide variety of medical procedures, including wound cleansing, post-surgery adhesion prevention, debris removal from surgical fields, etc., rely on the use of surgical irrigating solutions. While many advanced surgical procedures minimize damage to tissues compared to older techniques, certain delicate procedures remain very sensitive to techniques and materials used. In particular, ophthalmic surgical procedures, such as cataract surgery and vitrectomy surgery, involve very fragile tissues (such as the corneal endothelial layer) and accordingly have little room for error and great potential for harm to such ocular tissues and the vision of the patient. Many of these procedures rely on the use of surgical irrigating solutions to protect delicate ocular tissues from trauma. Thus, there is an ongoing need to improve ophthalmic surgical techniques and equipment, as well as associated pharmaceutical products such as surgical irrigating solutions, in order to minimize potential harm to ocular tissues during surgical procedures.

There are four key ingredients in surgical irrigating solutions for intraocular use: sodium chloride, a source of calcium, a source of magnesium and a source of bicarbonate anions. Sodium chloride is required to maintain the osmolality of the solution. Calcium ions are required to maintain the intercellular junctions in the corneal endothelium. Magnesium ions, like calcium ions, are found in the aqueous humor and are essential to a number of cellular processes. Bicarbonate anions are the physiological buffer for the eye that maintains the endothelial pump. Sodium chloride is compatible in almost any solution, so there is no issue with its use. However, calcium and magnesium can react with bicarbonate to form calcium or magnesium carbonates that precipitate out of the solution. This reaction happens immediately if a solution containing bicarbonate and calcium and/or magnesium is heat sterilized and may occur over time at ambient conditions. The reaction between calcium or magnesium and bicarbonate appears to occur with all ionic salts of calcium and magnesium typically used as pharmaceutical excipients.

BSS PLUS® Sterile Intraocular Irrigating Solution (Alcon Laboratories, Inc.) is a widely used ophthalmic surgical irrigating solution. BSS PLUS® Sterile Intraocular Irrigating Solution is a two-part solution; the parts are mixed together to form a single solution just prior to surgery. This mixing step can be inconvenient and potentially subject to error in a busy operating room. In addition, manufacturing two separate solutions is more complex and costly than manufacturing a one-part formulation; therefore a one-part irrigating solution is very desirable.

Part I of BSS PLUS® Sterile Intraocular Irrigating Solution contains sodium chloride, potassium chloride, sodium bicarbonate and dibasic sodium phosphate dissolved in water for injection. The pH of Part I is close to neutral, and it has an osmolality which is nearly isotonic with respect to physiological fluids. Part II of BSS PLUS® Sterile Intraocular Irrigating Solution contains calcium chloride, magnesium chloride, dextrose and glutathione disulfide (GSSG) dissolved in water for injection. The pH of Part II is adjusted to between 3 and 5 and the solution has an osmolality which is hypotonic. Divalent cations such as calcium and magnesium in Part II will react with bicarbonate and phosphate in Part I to form a precipitate if the two parts of BSS PLUS® Sterile Intraocular Irrigating Solution are combined. This reaction proceeds almost immediately if the combined solution is steam sterilized, but at room temperature occurs slowly over a period of several days. To prevent this precipitation, reconstituted BSS PLUS® Sterile Intraocular Irrigating Solution must be used within six hours after reconstitution (labeled shelf-life of the reconstituted product). Reconstituted BSS PLUS® Sterile Intraocular Irrigating Solution has a neutral pH and an osmolality which is isotonic.

In addition to forming precipitate as noted above, combining the two parts of BSS PLUS® Sterile Intraocular Irrigating Solution and terminally steam sterilizing the reconstituted solution can result in caramelization of dextrose and degradation of glutathione disulfide (GSSG).

There have been previous attempts to make a one-part irrigating solution comparable in performance to two-part BSS PLUS® Sterile Intraocular Irrigating Solution. EP1067907 B1 (Armitage, et al.) teaches the use of zwitterionic organic buffers such as N-(2-hydroxyethyl) piperazine-N′-(2-ethanesulfonic acid), commonly referred to as HEPES, to prevent the precipitation as discussed above. The formulations disclosed by Armitage, et al. do not contain components such as dextrose and GSSG that are known to be unstable when autoclaved or incorporated in physiological pH solutions. The formulations disclosed by Armitage, et al. also do not contain components of the type normally present in tissue culture media, such as amino acids. In contrast, embodiments of the present invention do not require the use of zwitterionic organic buffers such as HEPES, BES, MOPS, TES, EPPS, and TRICINE to maintain the solution in the physiological range. The teachings of Armitage, et al. do not appear to provide a solution to the problem of precipitate formation.

The stabilized irrigating solutions of the present invention solve these problems while being capable of withstanding terminal sterilization without precipitate formation.

### BRIEF SUMMARY OF THE INVENTION

The present invention relates in one embodiment to stabilized irrigating solutions comprising calcium glycerophosphate and/or sodium glycerophosphate, and one or more bicarbonate salts. Certain solutions of the present invention optionally comprise carbohydrate energy sources such as polysaccharides or monosaccharides, and may further optionally comprise glutathione disulfide. Preferred solutions are terminally sterilized. Preferred solutions comprising sodium glycerophosphate also comprise one or more calcium and/or magnesium salts.

Yet another embodiment of the present invention relates to methods of irrigating ocular tissues during a surgical procedure, which comprises bathing the intraocular tissues with a stabilized irrigating solution comprising calcium glycerophosphate and/or sodium glycerophosphate and one or more bicarbonate salts.

The foregoing brief summary broadly describes the features and technical advantages of certain embodiments of the present invention. Additional features and technical advantages will be described in the detailed description of the invention that follows. Novel features which are believed to be characteristic of the invention will be better understood from the detailed description of the invention when considered in connection with any accompanying figures. However, figures provided herein are intended to help illustrate the invention or assist with developing an understanding of the invention, and are not intended to be definitions of the invention's scope.

### BRIEF DESCRIPTION OF THE DRAWINGS

A more complete understanding of the present invention and the advantages thereof may be acquired by referring to the following description, taken in conjunction with the accompanying drawings and wherein:

FIG. 1 is a graph showing corneal perfusion data comparing BSS Sterile Intraocular Irrigating Solution to a calcium glycerophosphate formulation of the present invention.

### DETAILED DESCRIPTION OF THE INVENTION

The irrigating solutions of the present invention are generally aqueous, isotonic electrolyte solutions having a physiologically-compatible pH. The solutions are stabilized and thus do not become turbid or produce visible precipitates during a reasonable shelf life or following heat sterilization. The solutions comprise calcium glycerophosphate and/or sodium glycerophosphate and one or more bicarbonate salts.

Bicarbonate is a physiological buffer for the eye and bicarbonate salts are recognized as key components of ophthalmic irrigating solutions. Thus, embodiments of the present invention may include one or more bicarbonate salts at various concentrations including, without limitation, salts such as sodium or potassium bicarbonate. Preferred embodiments of the present invention comprise sodium bicarbonate. The concentration range of bicarbonate may be from about 0.1 w/v % to about 1.0 w/v % and the most preferred concentration is about 0.21 w/v %+10% (0.021 w/v %) excess.

It is desirable to include a calcium ion source in irrigating solutions for use in ophthalmic surgery, as calcium is found in the aqueous humor and is essential to a number of cellular processes, and increased calcium concentrations can prevent or reduce corneal edema during surgery. Magnesium is also found in the aqueous humor and is essential to a number of cellular processes. Magnesium is a required cofactor for a number of enzymes, some of which catalyze fatty acid synthesis, protein synthesis and glucose metabolism, and is involved in muscle contraction and relaxation, nerve impulse transmission and ATP metabolism. It is therefore desirable to include magnesium in surgical irrigating solutions as well. Certain solutions of the present invention comprise calcium glycerophosphate (CaGP) as a calcium source. Other solutions of the present invention comprise sodium glycerophosphate (NaGP), either by itself or in combination with CaGP, and optionally one or more calcium and/or magnesium salts as calcium and magnesium sources. The present inventors have made the unexpected discovery that irrigating solutions containing CaGP or NaGP, calcium, and magnesium salts and also containing bicarbonate are stabilized both at room temperature and following heat sterilization, and thus allow the use of higher calcium and magnesium ion concentrations.

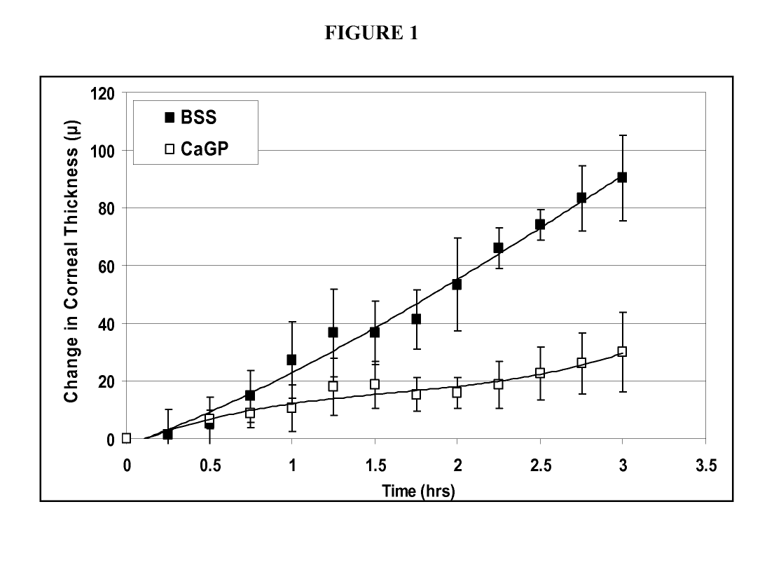

In addition to the stabilization of solutions containing bicarbonate, the properties of glycerophosphate are important with respect to other features of the present invention. First, calcium from CaGP appears to be available to the endothelial cells, based on perfusion data in FIG. 1 that shows reduced corneal edema compared to BSS® Sterile Irrigating Solution at every measured timepoint. Second, calcium and magnesium glycerophosphates (CaGP and MgGP) have negative solubility curves; i.e., they have lower solubilities at higher temperatures and higher solubilities at lower temperatures. When heat sterilized, solutions containing CaGP or NaGP and calcium and magnesium will precipitate CaGP or CaGP and MgGP as the bottle temperature increases. The solid CaGP and MgGP precipitates are not available for reaction with dissolved bicarbonate at high temperatures. However, when the solution returns to room temperature, CaGP and MgGP go back into solution. The above-described properties of CaGP and MgGP allow for increasing the concentrations of calcium and magnesium in an irrigating solution, thereby enhancing the ability of the solution to reduce edema and maintain normal cellular function. The concentrations of calcium and magnesium should be as high as possible without causing any precipitation. The preferred concentration of NaGP is from about 0.01 w/v % to about 0.5 w/v %, with a most preferred concentration of about 0.2 w/v %. The preferred concentration of calcium chloride is about 0.01 w/v % to about 0.5 w/v %, with a most preferred concentration of about 0.05 w/v %. The preferred concentration of magnesium chloride is about 0.01 w/v % to about 0.5 w/v %, with a most preferred concentration of about 0.02 w/v %. The preferred concentration of CaGP is from about 0.01 w/v % to about 0.5 w/v %, with a most preferred concentration of about 0.06 w/v %.

The solutions of the present invention may further comprise a buffering system to maintain pH. A variety of buffering systems known to those of skill in the art may be used with embodiments of the invention. However, in some embodiments of the present invention, bicarbonate by itself or in combination with other compounds provides adequate buffering capacity to maintain pH. Citrate buffers may also be used with certain embodiments of the present invention.

The solutions of the present invention may further comprise carbohydrate energy sources, such as polysaccharides, monosaccharides, sucrose, dextrose, etc. While dextrose may be used as an energy source in certain embodiments, if solutions comprising dextrose (such as BSS PLUS® Sterile Intraocular Irrigating Solution) are heat sterilized at a pH above 5, the dextrose tends to caramelize, forming an undesirable yellow color. Since an irrigating solution can have only one pH which must be close to a physiologic pH, it is difficult to prevent dextrose from caramelizing during sterilization unless the solution is pH-adjusted after the sterilization. Sucrose is believed to be more resistant to caramelization than the dextrose found in BSS PLUS® Sterile Intraocular Irrigating Solution. Thus, sucrose is a preferred polysaccharide energy source for use with certain embodiments of the present invention.

Histidine is an antioxidant and an essential amino acid that is present in preferred embodiments of the present invention. The use of histidine as a component of ocular irrigating solutions is described in co-pending U.S. application Ser. No. ______ (Attorney Docket No. 2787) entitled “INTRAOCULAR IRRIGATING SOLUTIONS AND METHODS FOR TREATING CORNEAL EDEMA,” filed on Dec. 19, 2008, the entire contents of which are hereby incorporated in the present specification by reference. Histidine also reduces the need for GSSG. Accordingly, GSSG is an optional ingredient of the solutions of the present invention. The concentration of histidine, if used, should be between about 0.1 w/v % to about 1.0 w/v %, but is preferably about 0.7 w/v %.

The solutions of the present invention may comprise additional essential ions such as sodium, potassium, and chloride. Potassium and sodium may be provided in the form of various sodium and potassium salts known to those of skill in the art, such as sodium or potassium chlorides, sulfates, acetates, citrates, lactates, and gluconates. Similarly, chloride salts, such as sodium chloride and potassium chloride, may be used to provide chloride in solutions of the present invention. For the essential ions, the concentration of potassium should be about 0.01 w/v % to about 0.5 w/v %, with the most preferred concentration about 0.04 w/v %. The concentration of sodium should be about 0.1 w/v % to about 1.0 w/v %, with the most preferred concentration about 0.55 w/v %.

The most preferred surgical irrigating solutions of the present invention contain NaGP; calcium and magnesium salts; bicarbonate as a physiological buffer; histidine as an antioxidant and to control corneal edema; essential ions such as sodium, potassium, and chloride; and optionally sucrose as an energy source.

As discussed above, a key advantage of certain formulations of the present invention is their ability to be terminally heat sterilized following addition and mixing of formulation ingredients. In preferred embodiments of the present invention, the formulation is prepared by mixing all ingredients and stirring until all components have entered solution. The solution is then sterilized by dry or steam heat for a set time period (typically 30 minutes at 121° C.). The time and temperature of sterilization may vary and can be easily optimized by those of skill in the art.

The irrigating solutions of the present invention are suitable for use in a variety of ophthalmic and non-ophthalmic surgical procedures, but are particularly adapted and well-suited for use in conjunction with ophthalmic surgical procedures. The solutions are especially useful in conjunction with anterior chamber ophthalmic procedures that have the potential to expose the endothelial cells of the cornea. In other applications, the solutions may be used for foreign body removal and washing procedures. The solutions are suitable for posterior chamber procedures such as vitrectomy and for procedures involving the retina. The above list is not comprehensive and those of skill in the art will appreciate other applications for the disclosed embodiments of the present invention.

The solutions described in Examples 1 and 2 below were prepared according to embodiments of the present invention and are provided to further illustrate various features of the present invention.

### Example 1

Formulation

| | |
|---|---|
| A | B |

| | |
|---|---|
| Ingredient | Concentration (% w/v) |

| | | |
|---|---|---|
| Sodium Chloride | 0.65 | 0.55 |
| Potassium Chloride | 0.1 | 0.04 |
| Sodium Citrate Dihydrate | 0.2 | 0.2 |
| Calcium Glycerophosphate, | 0.06 | 0.06 |
| hydrate | | |
| Sodium Bicarbonate | 0.21 + 10% | 0.21 + 10% |
| | excess | excess |
| Histidine | — | 0.7 |
| Water for Injection | qs 100 | qs 100 |
| F₀ for steam sterilization | 30 min. | 30 min. |
| Appearance | Clear | Clear |
| Increase in corneal thickness | 30 ± 14 μm | −16 ± 14 μm |
| with test solutions ^{a} | (n = 3) | (n = 2) |
| Increase in corneal thickness | 90 ± 15 μm | 15 ± 8 μm |
| with control solutions | (n = 3) ^{b} | (n = 2) ^{b} |

^{a} After 3 hours of perfusion; n = number of rabbit corneas.
^{b} BSS PLUS ® Sterile Intraocular Irrigating Solution.

### Example 2

| | Formulation | | | | |
|---|---|---|---|---|---|
| | C | D | E | F | G |
| Ingredient | Concentration (% w/v) | | | | |
| Sodium Chloride | 0.55 | 0.55 | 0.55 | 0.55 | 0.55 |
| Potassium Chloride | 0.04 | 0.04 | 0.04 | 0.04 | 0.04 |
| Calcium | 0.06 | — | — | — | — |
| Glycerophosphate | | | | | |
| Hydrate | | | | | |
| Disodium | — | 0.05 | 0.1 | 0.062 | 0.2 |
| Glycerophosphate | | | | | |
| Hydrate | | | | | |
| Calcium Chloride | — | 0.0154 | 0.031 | 0.042 | 0.062 |
| Dihydrate | | | | | |
| Magnesium Chloride | 0.02 | 0.02 | 0.02 | 0.02 | 0.02 |
| Hexahydrate | | | | | |
| Sodium Bicarbonate | 0.21 + 10% | 0.21 + 10% | 0.21 + 10% | 0.21 + 10% | 0.21 + 10% |
| | excess | excess | excess | excess | excess |
| Histidine | 0.7 | 0.7 | 0.7 | 0.7 | 0.7 |
| Sodium Citrate | 0.2 | 0.2 | 0.2 | 0.2 | 0.2 |
| Dihydrate | | | | | |
| Water for Injection | q.s. 100 | q.s. 100 | q.s. 100 | q.s. 100 | q.s. 100 |
| F₀ for steam | 30 min. | 30 min. | 30 min. | 30 min. | 30 min. |
| sterilization | | | | | |
| Appearance | Clear | Clear | Clear | Clear | Cloudy |

Corneal thickness changes in Example 1, formulations A and B were determined using the rabbit corneal perfusion model, in which paired corneas of New Zealand White rabbits were isolated and mounted in an in vitro dual-chambered specular microscope designed for endothelial perfusion evaluation. Corneal thickness readings were taken at 15-minute intervals with the specular microscope for the entire length of perfusion, which lasted for 3 three hours. The rabbit corneal perfusion model was also used to generate the data shown in FIG. 1, which shows a graph comparing the performance of formulation B compared to BSS PLUS® Sterile Intraocular Irrigating Solution. Formulation B is labeled as CaGP on the FIG. 1 graph.

The present invention and its embodiments have been described in detail. However, the scope of the present invention is not intended to be limited to the particular embodiments of any process, manufacture, composition of matter, compounds, means, methods, and/or steps described in the specification. Various modifications, substitutions, and variations can be made to the disclosed material without departing from the spirit and/or essential characteristics of the present invention. Accordingly, one of ordinary skill in the art will readily appreciate from the disclosure that later modifications, substitutions, and/or variations performing substantially the same function or achieving substantially the same result as embodiments described herein may be utilized according to such related embodiments of the present invention. Thus, the following claims are intended to encompass within their scope modifications, substitutions, and variations to processes, manufactures, compositions of matter, compounds, means, methods, and/or steps disclosed herein.

What is claimed is:

1. A stabilized irrigating composition comprising sodium glycerophosphate and/or calcium glycerophosphate, and a bicarbonate salt.

2. A composition of claim 1 comprising sodium glycerophosphate and further comprising calcium and magnesium salts.

3. A composition of claim 1, wherein the bicarbonate salt is selected from the group consisting of:
sodium bicarbonate, potassium bicarbonate, and combinations thereof.

4. A composition of claim 1, further comprising glutathione disulfide and/or histidine.

5. A composition of claim 1, further comprising a carbohydrate energy source selected from the group consisting of:
polysaccharides, monosaccharides, and combinations thereof.

6. A composition of claim 5, wherein said energy source is selected from the group consisting of:
sucrose, dextrose, and combinations thereof.

7. A composition of claim 1, wherein the concentration of said bicarbonate is about 0.1 w/v % to about 1.0 w/v %.

8. A composition of claim 1, wherein the concentration of said bicarbonate is about 0.21 w/v % plus an excess of 10% (0.021 w/v %).

9. A composition of claim 1, comprising sodium glycerophosphate at a concentration of about 0.01 w/v % to about 0.5 w/v %.

10. A composition of claim 1, comprising sodium glycerophosphate at a concentration of about 0.2 w/v %.

11. A composition of claim 2, wherein a calcium salt is calcium chloride at a concentration of about 0.01 w/v % to about 0.5 w/v % and wherein a magnesium salt is magnesium chloride at a concentration of about 0.01 w/v % to about 0.5 w/v %.

12. A composition of claim 11, wherein the concentration of said calcium chloride is about 0.05 w/v % and the concentration of said magnesium chloride is about 0.02 w/v %.

13. A composition of claim 1, comprising calcium glycerophosphate at a concentration of about 0.01 w/v % to about 0.5 w/v %.

14. A composition of claim 1, comprising calcium glycerophosphate at a concentration of about 0.06 w/v %.

15. A composition of claim 4, comprising histidine at a concentration of about 0.1 w/v % to about 1.0 w/v %.

16. A composition of claim 4, comprising histidine at about 0.7 w/v %.

17. A composition of claim 1, further comprising potassium chloride at a concentration of about 0.01 w/v % to about 0.5 w/v % and sodium chloride at a concentration of about 0.1 w/v % to about 1.0 w/v %.

18. A composition of claim 17, comprising potassium chloride at about 0.04 w/v % and sodium chloride at a concentration of about 0.55 w/v %.

19. A method of irrigating ocular tissues during a surgical procedure, which comprises bathing the intraocular tissues with an irrigating composition according to claim 1.

20. A method of claim 19 wherein said irrigating composition comprises sodium glycerophosphate and further comprises calcium and magnesium salts.
